(19) United States
(12) Patent Application Publication
(10) Pub. No.: US 2025/0308024 A1
(43) Pub. Date: Oct. 2, 2025

(54) IMAGE PROCESSING DEVICE, IMAGE PROCESSING METHOD, IMAGE PROCESSING PROGRAM, LEARNING DEVICE, LEARNING METHOD, AND LEARNING PROGRAM

(71) Applicant: FUJIFILM CORPORATION, Tokyo (JP)

(72) Inventor: Tatsuki KOIKE, Tokyo (JP)

(21) Appl. No.: 19/082,192

(22) Filed: Mar. 18, 2025

(30) Foreign Application Priority Data
Mar. 26, 2024 (JP) 2024-049357

(51) Int. Cl. G06T 7/00; G06T 7/73; G06T 7/77

(52) U.S. Cl. CPC G06T 7/0014; G06T 7/74; G06T 7/75; G06T 7/77; G06T 2207/10072; G06T 2207/20081; G06T 2207/20084; G06T 2207/30056; G06T 2207/30204

(57) ABSTRACT

An image processing device includes a processor, in which the processor is configured to: input at least one processing target tomographic image to a derivation model constructed by contrastive learning using a plurality of tomographic images acquired by imaging an interior of a body such that a specific anatomical structure is included, the derivation model being constructed by the contrastive learning so as to derive a normalized relative position in the interior of the body based on a relative reference position, which is determined in advance for the specific anatomical structure, in the interior of the body; and derive a normalized relative position of the at least one processing target tomographic image in the interior of the body via the derivation model.

### CROSS REFERENCE TO RELATED APPLICATIONS

The present application claims priority from Japanese Patent Application No. 2024-049357, filed on Mar. 26, 2024, the entire disclosure of which is incorporated herein by reference.

### BACKGROUND

### Technical Field

The present disclosure relates to an image processing device, an image processing method, an image processing program, a learning device, a learning method, and a learning program.

### Related Art

In recent years, with the advancement of medical equipment, such as a computed tomography (CT) apparatus and a magnetic resonance imaging (MRI) apparatus, three-dimensional images having a higher quality and a higher resolution have been used for image diagnosis.

In a case in which a subject is imaged by using an imaging apparatus, such as the CT apparatus or the MRI apparatus, in order to determine an imaging range, scout imaging is performed before main imaging for acquiring a three-dimensional image to acquire a two-dimensional image for positioning (scout image). An operator of an imaging apparatus, such as a technician, sets the imaging range at the time of main imaging while viewing the scout image.

Meanwhile, since the operator needs to perform the setting manually, the setting of the imaging range while viewing the scout image requires time. In addition, since the setting accuracy depends on the ability and the experience of the operator, there is a variation in the setting accuracy. Therefore, various methods for automatically setting the imaging range from the scout image have been proposed (for example, see Ruiqi Geng MSc, et al, Automated MR Image Prescription of the Liver Using Deep Learning: Development, Evaluation, and Prospective Implementation, 30 Dec. 2022).

However, the scout image has a larger slice interval than the three-dimensional image acquired by the main imaging and has a smaller number of tomographic images than the three-dimensional image. Therefore, a situation may occur in which the tomographic image included in the scout image does not include a target anatomical structure. In this case, it is considered to set the imaging range with reference to other anatomical structures included in the tomographic image. However, in a case in which the other anatomical structures are not included in the tomographic image, it is not possible to specify the position of the target anatomical structure, and, as a result, it is not possible to set the imaging range.

### SUMMARY OF THE INVENTION

The present disclosure has been made in view of the above-described circumstances, and an object of the present disclosure is to enable specification of a position of a target anatomical structure based on a tomographic image such as a scout image even in a case in which the target anatomical structure is not included in the tomographic image.

The present disclosure provides an image processing device comprising: a processor, in which the processor is configured to: input at least one processing target tomographic image to a derivation model constructed by contrastive learning using a plurality of tomographic images acquired by imaging an interior of a body such that a specific anatomical structure is included, the derivation model being constructed by the contrastive learning so as to derive a normalized relative position in the interior of the body based on a relative reference position, which is determined in advance for the specific anatomical structure, in the interior of the body; and derive a normalized relative position of the at least one processing target tomographic image in the interior of the body via the derivation model.

The present disclosure provides a learning device comprising: a processor, in which the processor is configured to: train a learning target model through contrastive learning so as to derive, in a case in which a plurality of tomographic images including a specific anatomical structure is input, a normalized relative position in an interior of a body based on a relative reference position, which is determined in advance for the specific anatomical structure, in the interior of the body, to construct a derivation model that derives, in a case in which at least one processing target tomographic image is input, a normalized relative position of the at least one processing target tomographic image in the interior of the body.

The present disclosure provides an image processing method executed by a computer, the image processing method including: inputting at least one processing target tomographic image to a derivation model constructed by contrastive learning using a plurality of tomographic images acquired by imaging an interior of a body such that a specific anatomical structure is included, the derivation model being constructed by the contrastive learning so as to derive a normalized relative position in the interior of the body based on a relative reference position, which is determined in advance for the specific anatomical structure, in the interior of the body; and deriving a normalized relative position of the at least one processing target tomographic image in the interior of the body via the derivation model.

The present disclosure provides a learning method executed by a computer, the learning method including: training a learning target model through contrastive learning so as to derive, in a case in which a plurality of tomographic images including a specific anatomical structure is input, a normalized relative position in an interior of a body based on a relative reference position, which is determined in advance for the specific anatomical structure, in the interior of the body, to construct a derivation model that derives, in a case in which at least one processing target tomographic image is input, a normalized relative position of the at least one processing target tomographic image in the interior of the body.

The present disclosure provides an image processing program causing a computer to execute a procedure including: inputting at least one processing target tomographic image to a derivation model constructed by contrastive learning using a plurality of tomographic images acquired by imaging an interior of a body such that a specific anatomical structure is included, the derivation model being constructed by the contrastive learning so as to derive a normalized relative position in the interior of the body based on a relative reference position, which is determined in advance for the specific anatomical structure, in the interior of the body; and deriving a normalized relative position of the at least one processing target tomographic image in the interior of the body via the derivation model.

The present disclosure provides a learning program causing a computer to execute a procedure including: training a learning target model through contrastive learning so as to derive, in a case in which a plurality of tomographic images including a specific anatomical structure is input, a normalized relative position in an interior of a body based on a relative reference position, which is determined in advance for the specific anatomical structure, in the interior of the body, to construct a derivation model that derives, in a case in which at least one processing target tomographic image is input, a normalized relative position of the at least one processing target tomographic image in the interior of the body.

According to the present disclosure, even in a case in which the target anatomical structure is not included in the tomographic image, the position of the target anatomical structure can be specified based on the tomographic image.

### BRIEF DESCRIPTION OF THE DRAWINGS

FIG. 1 is a diagram showing a schematic configuration of a medical information system to which an image processing device and a learning device according to an embodiment of the present disclosure are applied.

FIG. 2 is a diagram showing a schematic configuration of the image processing device and the learning device according to the present embodiment.

FIG. 3 is a functional configuration diagram of the image processing device and the learning device according to the present embodiment.

FIG. 4 is a diagram showing an example of a three-dimensional image used during learning.

FIG. 5 is a diagram showing an example of training data.

FIG. 6 is a diagram showing training of a CNN for constructing a derivation model.

FIG. 7 is a diagram showing normalization in an interior of a body.

FIG. 8 is a diagram showing training of the CNN for constructing the derivation model.

FIG. 9 is a diagram showing processing performed by a derivation unit.

FIG. 10 is a diagram showing derivation of a range of a scout image.

FIG. 11 is a diagram showing a display screen.

FIG. 12 is a flowchart showing processing performed by the learning device in the present embodiment.

FIG. 13 is a flowchart showing processing performed by the image processing device in the present embodiment.

### DETAILED DESCRIPTION

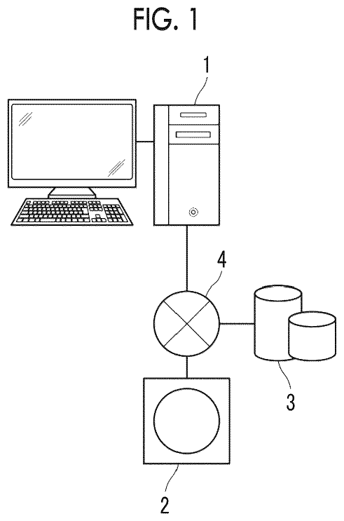

Hereinafter, an embodiment of the present disclosure will be described with reference to the drawings. First, a configuration of a medical information system to which an image processing device and a learning device according to the present embodiment are applied will be described. FIG. 1 is a diagram showing a schematic configuration of the medical information system. In the medical information system shown in FIG. 1, a computer 1 including the image processing device and the learning device according to the present embodiment, an imaging apparatus 2, and an image storage server 3 are connected via a network 4 in a communicable state.

The computer 1 includes the image processing device and the learning device according to the present embodiment, and an image processing program and a learning program according to the present embodiment are installed in the computer 1. The computer 1 may be a workstation or a personal computer directly operated by a doctor who makes a diagnosis, or may be a server computer connected to the workstation or the personal computer via the network. The image processing program is stored in a storage device of the server computer connected to the network or in a network storage to be accessible from the outside, and is, in response to a request, downloaded and installed in the computer 1 used by the doctor. Alternatively, the image processing program is distributed in a state of being recorded on a recording medium, such as a digital versatile disc (DVD) or a compact disc read-only memory (CD-ROM), and is installed in the computer 1 from the recording medium.

The imaging apparatus 2 is an apparatus that generates a two-dimensional image or a three-dimensional image representing a part of a subject to be diagnosed by imaging the part, and is specifically a radiography apparatus, a computed tomography (CT) apparatus, a magnetic resonance imaging (MRI) apparatus, a positron emission tomography (PET) apparatus, or the like. The image of the subject generated by the imaging apparatus 2 is transmitted to the image storage server 3 and stored in the image storage server 3. It should be noted that the three-dimensional image includes a plurality of tomographic images or an image composed of three-dimensional coordinates generated from the plurality of tomographic images.

The image storage server 3 is a computer that stores and manages various types of data, and comprises a large-capacity external storage device and software for database management. The image storage server 3 communicates with another device via the wired or wireless network 4, and transmits and receives image data and the like to and from the other device. Specifically, the image storage server 3 acquires various types of data including the image data of the image generated by the imaging apparatus 2 via the network, and stores and manages the various types of data in the recording medium, such as the large-capacity external storage device. It should be noted that a storage format of the image data and the communication between the devices via the network 4 are based on a protocol such as digital imaging and communication in medicine (DICOM).

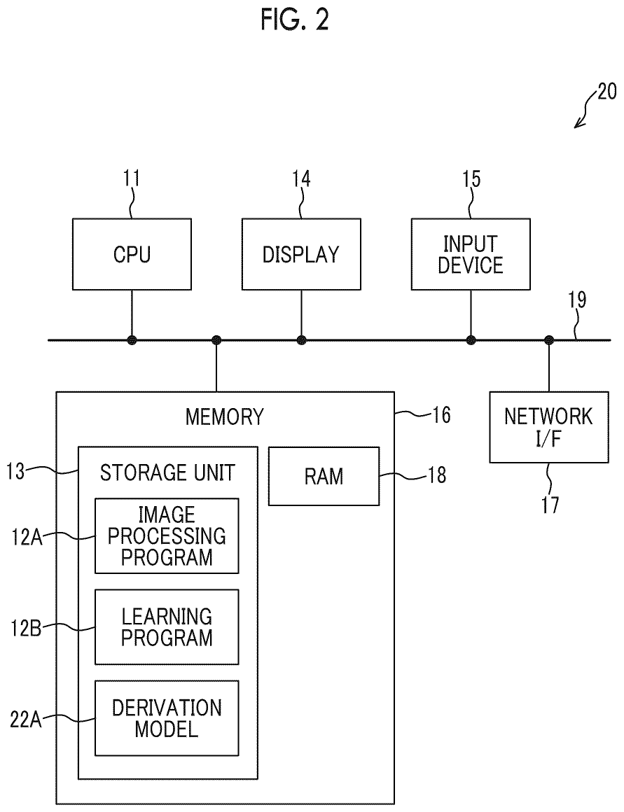

Next, the image processing device and the learning device according to the present embodiment will be described. It should be noted that, in the following description, the image processing device and the learning device may be represented only by the image processing device. FIG. 2 is a diagram showing a hardware configuration of the image processing device according to the present embodiment. As shown in FIG. 2, the image processing device 20 includes a central processing unit (CPU) 11, a display 14, an input device 15, a memory 16, and a network interface (I/F) 17 connected to the network 4. The CPU 11, the display 14, the input device 15, the memory 16, and the network I/F 17 are connected to a bus 19. It should be noted that the CPU 11 is an example of a processor in the present disclosure.

The memory 16 includes the storage unit 13 and a random access memory (RAM) 18. The RAM 18 is a primary storage memory, and is, for example, a RAM such as a static random access memory (SRAM) or a dynamic random access memory (DRAM).

The storage unit 13 is a non-volatile memory and is implemented by, for example, at least one of a hard disk drive (HDD), a solid state drive (SSD), an electrically erasable and programmable read only memory (EEPROM), or a flash memory. The storage unit 13 as a storage medium stores an image processing program 12A and a learning program 12B according to the present embodiment. The CPU 11 reads out the image processing program 12A and the learning program 12B from the storage unit 13, loads the image processing program 12A and the learning program 12B in the RAM 18, and executes the loaded image processing program 12A and learning program 12B. It should be noted that the storage unit 13 also stores a derivation model 22A described below.

The display 14 is a device that displays various screens, and is, for example, a liquid crystal display or an electro luminescence (EL) display. The input device 15 is a device for a user to perform input, and is, for example, at least any one of a keyboard, a mouse, a microphone for audio input, a touchpad for proximity input including contact, or a camera for gesture input. The network I/F 17 is an interface for connection to the network 4.

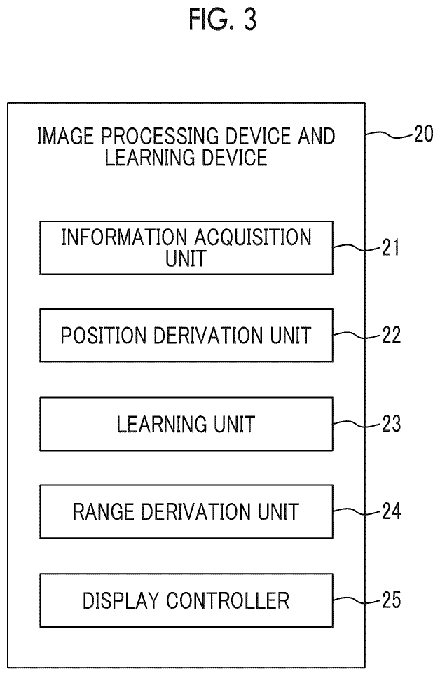

Hereinafter, a functional configuration of the image processing device according to the present embodiment will be described. FIG. 3 is a diagram showing a functional configuration of the image processing device and the learning device according to the present embodiment. As shown in FIG. 3, the image processing device 20 comprises an information acquisition unit 21, a position derivation unit 22, a learning unit 23, a range derivation unit 24, and a display controller 25. In a case in which the CPU 11 executes the image processing program 12A, the CPU 11 functions as the information acquisition unit 21, the position derivation unit 22, the range derivation unit 24, and the display controller 25. In a case in which the CPU 11 executes the learning program 12B, the CPU 11 functions as the learning unit 23.

The information acquisition unit 21 acquires a medical image that is a processing target from the image storage server 3 in response to an instruction from the operator through the input device 15. In the present embodiment, the medical image is a scout image G0 used for positioning during the imaging using the CT apparatus or during the imaging using the MRI apparatus. The scout image includes a plurality of tomographic images, has a larger slice interval than the three-dimensional image, and has a smaller number of tomographic images than the three-dimensional image. The tomographic image included in the scout image G0 is an example of a processing target tomographic image according to the present disclosure.

In addition, the information acquisition unit 21 acquires training data used to train a derivation model, which will be described below, from the image storage server 3. The training data will be described below.

The position derivation unit 22 inputs at least one processing target image included in the scout image G0 to the derivation model 22A, and derives a normalized relative position of the processing target tomographic image in the interior of the body. It should be noted that the relative position of the processing target tomographic image in the interior of the body is a relative position of any point determined in advance on the processing target tomographic image in the interior of the body. Any point can be used as the center point and the points of the four corners of the processing target tomographic image, but the present disclosure is not limited to this. In the present embodiment, the relative position of the processing target tomographic image is a relative position of the center point of the processing target tomographic image in the interior of the body.

The derivation model 22A is constructed by using, as training data, a plurality of tomographic images acquired by imaging the interior of the body such that a specific anatomical structure is included, and training, for example, a convolutional neural network (CNN) through contrastive learning. The CNN is an example of a learning target model according to the present disclosure. The contrastive learning is learning of making a distance between feature values derived from the same image close to each other in a feature value space and making a distance between feature values derived from different images far from each other in the feature value space. As the contrastive learning, for example, a learning method such as A Simple Framework for Contrastive Learning of Visual Representations (SimCLR) is known.

In the present embodiment, for example, a method described in “Dewen Zeng, et.al, Positional Contrastive Learning for Volumetric Medical Image Segmentation, arXiv: 2106.09157, 16 Jun. 2021” is used to perform the contrastive learning to derive the normalized relative position in the interior of the body based on a relative reference position in the interior of the body, which is determined in advance for the specific anatomical structure, so that the derivation model 22A is constructed.

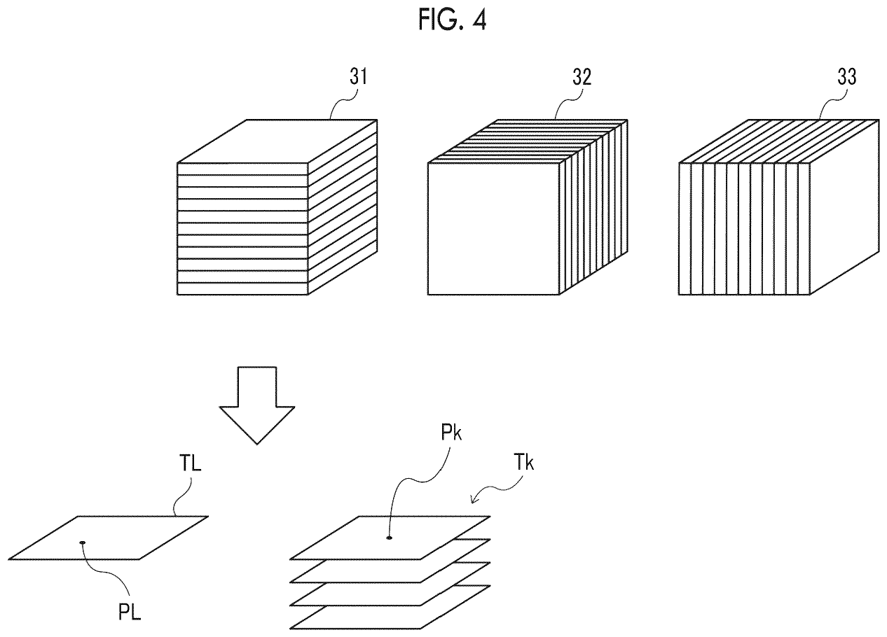

The learning unit 23 constructs the derivation model 22A by training the CNN through the contrastive learning as described above. In the training of the CNN, the tomographic images included in the three-dimensional image are used as the training data. FIG. 4 is a diagram showing an example of the three-dimensional image used for the learning. In the present embodiment, a three-dimensional image 31 of an axial cross section acquired by imaging a human body is used to train the CNN, but a three-dimensional image 32 of a coronal cross section and a three-dimensional image 33 of a sagittal cross section may be used to train the CNN instead of or in addition to the three-dimensional image 31 of the axial cross section. It should be noted that the three-dimensional image including the tomographic image used as the training data is referred to as a three-dimensional image for training.

The three-dimensional image for training is acquired such that the specific anatomical structure is included. As the specific anatomical structure, a landmark such as an upper end of a liver in the interior of the body or a center of a specific vertebra can be used. In the present embodiment, the specific anatomical structure is the upper end of the liver.

In the learning, the learning unit 23 uses a plurality of tomographic images (referred to as tomographic images for training) Tk (k=1 to n: n is the number of tomographic images) included in the three-dimensional image for training 31 as the training data. In addition, a tomographic image for training TL including a landmark PL among the plurality of tomographic images for training Tk is also used as the training data. The tomographic image for training including the landmark PL is referred to as a reference tomographic image for training TL.

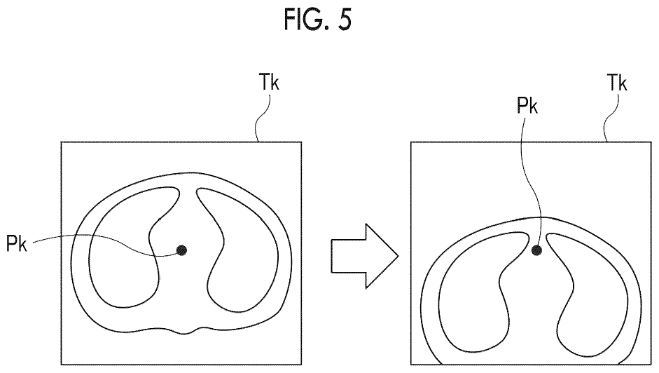

It should be noted that, as shown in FIG. 5, the learning unit 23 may derive a new tomographic image for training Tk by shifting a subject region included in the tomographic image for training Tk in the tomographic image plane. Therefore, in the learning, the reference tomographic image for training TL in which the center point is the landmark PL may be used.

The learning unit 23 derives a relative positional relationship between the center points Pk in any two tomographic images for training extracted from the plurality of tomographic images for training Tk, and uses the relative positional relationship as ground truth data during the learning. The relative positional relationship is derived in each of three axial directions, that is, the x-direction, the y-direction, and the z-direction in the three-dimensional image for training 31. For example, as the relative positional relationship, ground truth data is derived, which indicates that a center point P2 of a tomographic image for training T2 is on a plus side in the x-direction, on a minus side in the y-direction, and on a plus side in the z-direction with respect to a center point P1 of a tomographic image for training T1.

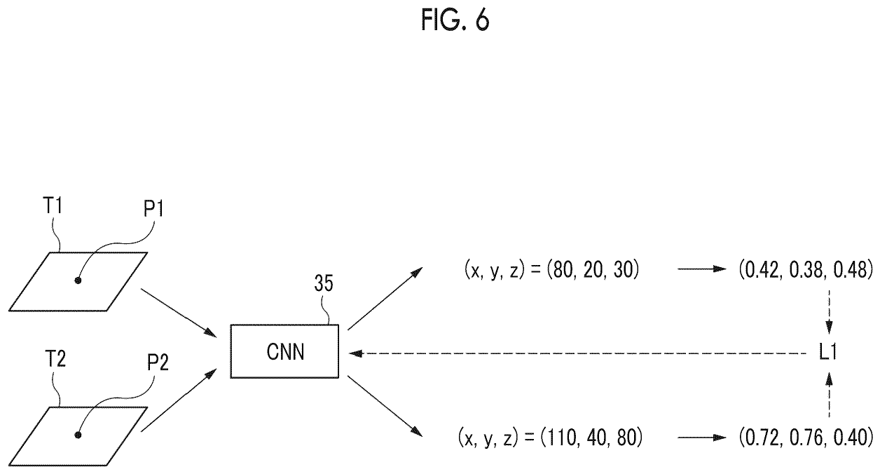

FIG. 6 is a diagram showing training of the CNN for constructing the derivation model. In the learning shown in FIG. 6, it is assumed that two tomographic images for training T1 and T2, which do not include the landmark, are used as the training data. In FIG. 6, the learning unit 23 inputs the two tomographic images for training T1 and T2 to the CNN 35.

The CNN 35 outputs a median value representing the positions of the center points P1 and P2 of the two input tomographic images for training T1 and T2 in the interior of the body. The median value is coordinate values in the x-direction, the y-direction, and the z-direction, but can be recognized only in the internal processing of the CNN 35, and is a value with the origin in the internal processing of the CNN 35 as a reference. In FIG. 6, it is assumed that (80, 20, 30) is derived as the median value representing the position of the center point P1 of the tomographic image for training T1, and (110, 40, 80) is derived as the median value representing the position of the center point P2 of the tomographic image for training T2.

The CNN 35 applies a sigmoid function to the median value so that the coordinate values of x, y, and z are values of 0 or more and 1 or less, and outputs the position coordinates of the normalized center points P1 and P2. It is assumed that, by the normalization, the position coordinates of the center point P1 are (0.42, 0.38, 0.48), and the position coordinates of the center point P2 are (0.72, 0.76, 0.82). The position coordinates of the normalized center points P1 and P2 represent the relative positions of the center points P1 and P2 in the tomographic images for training T1 and T2 in the interior of the body.

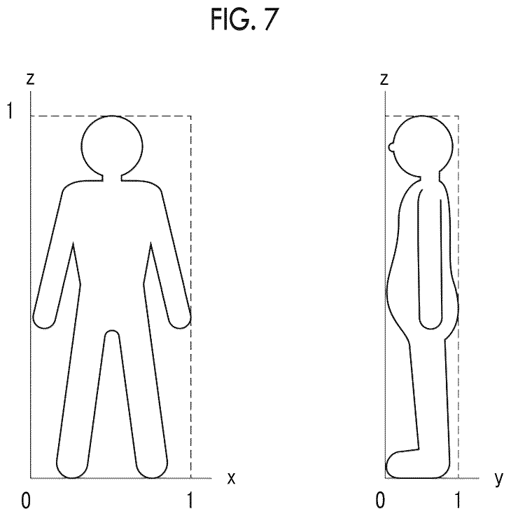

FIG. 7 is a diagram showing the normalization in the interior of the body. As shown in FIG. 7, in a coronal direction (x-direction), the x-coordinate value is normalized so that the position between the right and left end parts of the human body is 0 or more and 1 or less. The right and left end parts of the human body can be set to a range from an end part of a right arm to an end part of a left arm. In a sagittal direction (y-direction), the y-coordinate value is normalized so that the position between the front and back end parts of the human body is 0 or more and 1 or less. The front and back end parts of the human body can be set to a range from a most protruding position (for example, an abdomen) on the front surface of the human body to a most protruding position on the back surface (for example, a buttock) of the human body. In an axial direction (z-direction), the z-coordinate value is normalized so that the range of the height, that is, the position between the sole of the foot and the top of the head is 0 or more and 1 or less.

For the tomographic image for training T1, the position coordinates of the normalized center point P1 are (0.42, 0.38, 0.48). Therefore, the center point P1 of the tomographic image for training T1 is located at a position of 0.42 from the end part of the right arm in a case in which a distance between the right and left end parts is 1, is located at a position of 0.38 from the most protruding position of the abdomen in a case in which a distance between the front and back end parts is 1, and is located at a position of 0.48 from the sole of the foot in a case in which a height is 1.

It should be noted that, since the interior of the body is normalized as shown in FIG. 7, any position in the interior of the body can be represented by a normalized relative position. The top of the head can be represented by, for example, (0.5, 0.5, 1.0), and the upper end of the liver can be represented by, for example, (0.50, 0.50, 0.60).

The learning unit 23 derives a difference in relative positional relationship between the center points P1 and P2 of the two tomographic images for training T1 and T2 in the interior of the body as a first loss L1. The position coordinates of the normalized center point P1 of the tomographic image for training T1 are (0.42, 0.38, 0.48), and the position coordinates of the normalized center point P2 of the tomographic image for training T2 are (0.72, 0.76, 0.40). This represents that the center point P2 of the tomographic image for training T2 derived by the CNN 35 is on the plus side in the x-direction, on the plus side in the y-direction, and on the minus side in the z-direction with respect to the center point P1 of the tomographic image for training T1.

In a case in which the ground truth data for the center points P1 and P2 is on the plus side in the x-direction, on the minus side in the y-direction, and on the plus side in the z-direction, the CNN 35 correctly outputs the positional relationship in the x-direction, and thus the first loss L1 is 0. On the other hand, since the positional relationship in the y-direction and the z-direction is incorrect, the first loss L1 in the y-direction and the z-direction is generated.

The learning unit 23 trains the CNN 35 by performing, as appropriate, weighting on the first loss L1 so that the first loss L1 is 0 in all of the x-direction, the y-direction, and the z-direction.

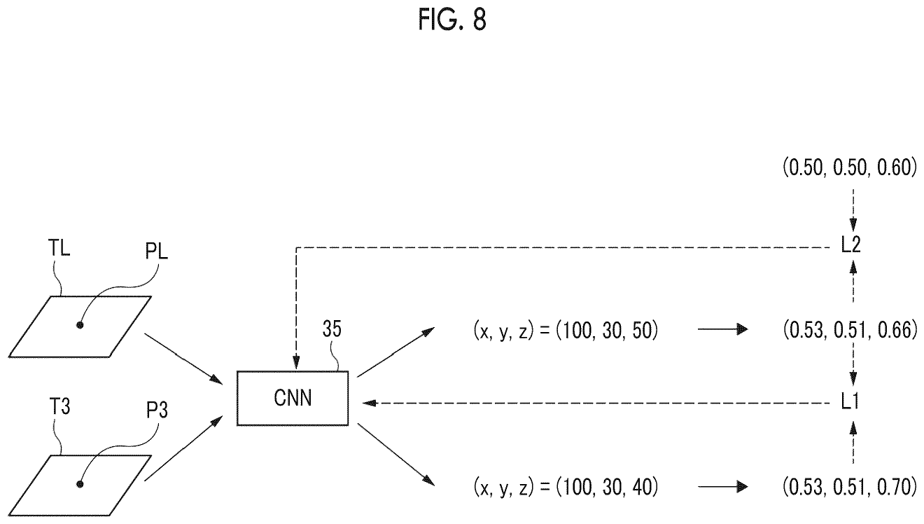

FIG. 8 is a diagram showing training of the CNN for constructing the derivation model using the training data different from that of FIG. 6. In the learning shown in FIG. 8, as the training data, the reference tomographic image for training TL in which the center point is the landmark PL and a tomographic image for training T3 that does not include the landmark are used. The learning unit 23 inputs the reference tomographic image for training TL and the tomographic image for training T3 to the CNN 35.

The CNN 35 outputs the median value representing the positions of the center points PL and P3 of the input reference tomographic image for training TL and tomographic image for training T3 in the interior of the body. In FIG. 8, it is assumed that (100, 30, 50) is derived as the median value representing the position of the center point PL of the reference tomographic image for training TL, and (100, 30, 40) is derived as the median value representing the position of the center point P3 of the tomographic image for training T3.

The CNN 35 normalizes the median value, and outputs the position coordinates of the normalized center point PL and center point P3. It is assumed that, by the normalization, the position coordinates of the center point PL of the reference tomographic image for training TL are (0.53, 0.51, 0.66), and the position coordinates of the center point P3 of the tomographic image for training T3 are (0.53, 0.51, 0.70).

The learning unit 23 derives a difference in relative positional relationship between the reference tomographic image for training TL and the tomographic image for training T3 in the interior of the body as the first loss L1. The position coordinates of the normalized center point PL of the reference tomographic image for training TL are (0.53, 0.51, 0.66), and the position coordinates of the normalized center point P3 of the tomographic image for training T3 are (0.53, 0.51, 0.70). This represents that the center point P3 matches the center point PL in the x-direction and the y-direction, and is located on the plus side in the z-direction.

In a case in which the ground truth data for the center points PL and P3 is 0 in the x-direction, 0 in the y-direction, and on the minus side in the z-direction, the CNN 35 correctly outputs the positional relationship in the x-direction and the y-direction, so that the first loss L1 is 0. On the other hand, since the positional relationship in the z-direction is incorrect, the first loss L1 in the y-direction and the z-direction is generated.

On the other hand, in a case in which the learning unit 23 uses the reference tomographic image for training TL for learning, the learning unit 23 derives a difference between normalized coordinate values of the center point PL of the reference tomographic image for training TL and normalized coordinate values of a reference position of the predetermined specific anatomical structure as a second loss L2. In the present embodiment, the specific anatomical structure is the upper end of the liver. In the present embodiment, the normalized coordinate values of the upper end of the liver in the interior of the body are derived in advance. For example, the coordinate values of the normalized reference position of the upper end of the liver are derived as (0.50, 0.50, 0.60). Therefore, the learning unit 23 derives, as the second loss L2, a difference between the normalized coordinate values (0.53, 0.51, 0.66) of the center point PL derived by the CNN 35 for the reference tomographic image for training TL and the normalized coordinate values (0.50, 0.50, 0.60) as a reference. As the difference, for example, a least square error can be used, but the present disclosure is not limited to this.

The learning unit 23 trains the CNN 35 by performing, as appropriate, weighting on the first loss L1 and the second loss L2 so that the first loss L1 is 0 and the second loss L2 is equal to or less than a predetermined threshold value.

As the learning progresses, the CNN 35 can output the normalized relative position of the center point of the input tomographic image in the x-direction, the y-direction, and the z-direction in the interior of the body. In addition, in a case in which the upper end of the liver is included in the input tomographic image, the position in the interior of the body can be output, which is normalized so that the position coordinates of the position to be output for the upper end of the liver are (0.50, 0.50, 0.60). By advancing the learning in this way, the CNN 35 is constructed as the derivation model 22A.

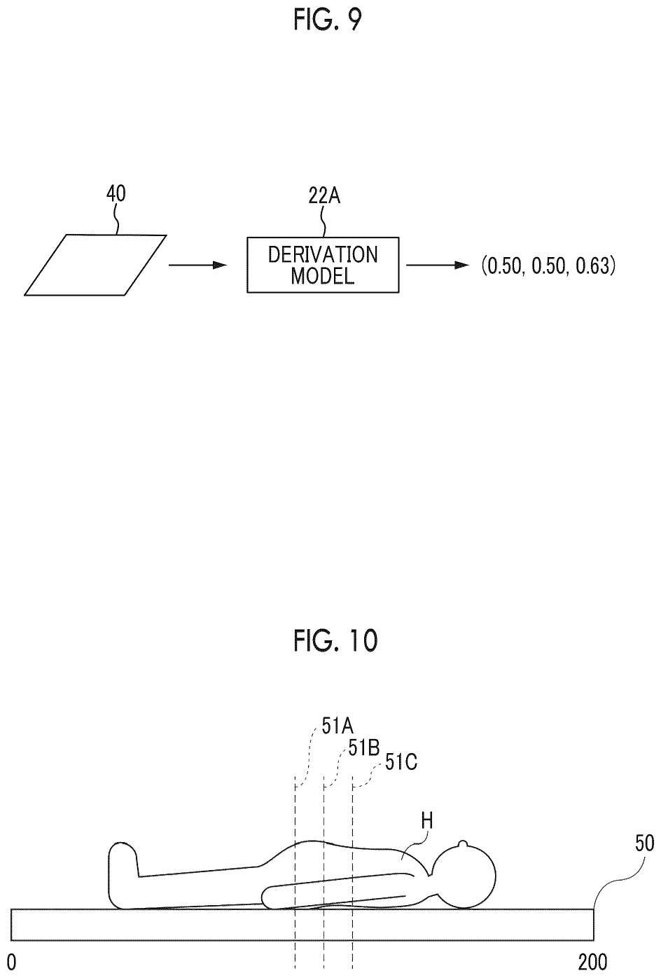

FIG. 9 is a diagram showing processing performed by the derivation model 22A constructed as described above. As shown in FIG. 9, in a case in which one processing target tomographic image 40 included in the scout image G0 is input to the derivation model 22A, the relative position of the processing target tomographic image 40 in the interior of the body is output from the derivation model 22A. In FIG. 9, (0.50, 0.50, 0.63) is output as an example. Therefore, by inputting all the processing target tomographic images included in the scout image G0 to the derivation model 22A, the relative positions of all the processing target tomographic images in the interior of the body can be derived.

The range derivation unit 24 derives the imaging range of the scout image. For this purpose, the range derivation unit 24 derives an absolute position of the imaging range of the scout image with the imaging apparatus 2 as a reference, for the relative position, in the interior of the body, of the processing target tomographic image derived by the position derivation unit 22. As the reference position in the imaging apparatus 2, for example, a position of an end part of an examination table on which a subject lies can be used. FIG. 10 is a diagram showing the derivation of the absolute position of the scout image. In the imaging apparatus 2, an absolute coordinate in a longitudinal direction of an examination table 50 on which the subject lies is acquired. For example, as shown in FIG. 10, the absolute coordinates are derived such that an end part of the examination table 50 on the leg side of the subject H is 0, and an end part of the examination table 50 on the head side is 200. In addition, the absolute coordinates of positions 51A to 51C of the examination table in the longitudinal direction of the tomographic images acquired by the imaging are also acquired during the imaging. Therefore, the relative coordinate and the absolute coordinate in the z-direction for the relative position derived by the position derivation unit 22 can be associated with each other, and the range derivation unit 24 can derive the absolute coordinate from the relative coordinate in the z-direction of the derived scout image and can specify the imaging range of the scout image based on the absolute coordinate.

Here, as shown in FIG. 7, since the position in the interior of the body is normalized, it is possible to recognize the organ in the interior of the body that is included the input tomographic image by referring to the relative position, in the interior of the body, of the processing target tomographic image output by the derivation model 22A. For example, in a case in which the liver is set as an imaging target, it is possible to determine whether or not the liver is included in the scout image by referring to the relative position of the tomographic image in the interior of the body included in the scout image. On the other hand, even in a case in which the scout image does not include the liver, the position of the liver with respect to the acquired scout image can be recognized by comparing the relative position, in the interior of the body, of the tomographic image included in the scout image with the relative position of the liver. In this way, by associating the derived relative position in the interior of the body with the absolute position, it is possible to specify the imaging range of the scout image, and it is possible to specify the imaging range during the main imaging such that the target anatomical structure is included, based on the imaging range of the scout image.

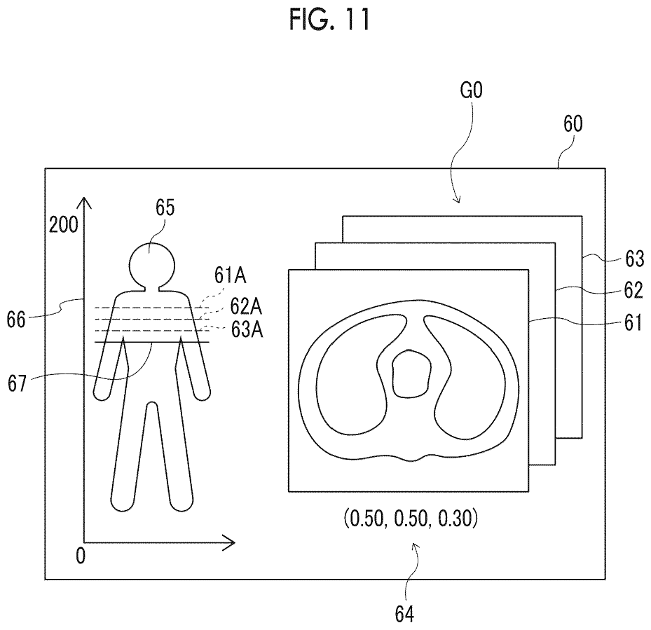

The display controller 25 displays the imaging range derived by the range derivation unit 24 together with the tomographic image. FIG. 11 is a diagram showing a display screen. It should be noted that, here, it is assumed that the input scout image G0 includes three tomographic images of the axial cross section. As shown in FIG. 11, processing target tomographic images 61 to 63 included in the input scout image G0 are displayed on a display screen 60 in a switchable manner. A relative position 64 (here, (0.50, 0.50, 0.30)) in the interior of the body for the displayed processing target tomographic image is displayed below the processing target tomographic images 61 to 63. Further, a schematic diagram 65 of the human body is displayed on the display screen 60. Lines 61A to 63A representing the positions of the processing target tomographic images 61 to 63 in the axial direction are displayed in the schematic diagram 65. A range between the line 61A and the line 63A is the imaging range of the scout image G0. In addition, a line 67 indicating a position of the upper end of the liver, which is the landmark, is displayed in the schematic diagram 65. In the present embodiment, the relative position of the processing target tomographic image in the interior of the body is derived, and the absolute position is further derived from the relative position to derive the imaging range. Therefore, the absolute coordinates 66 in the axial direction are also displayed on the display screen 60.

Here, in a case in which the imaging target is the liver, in the display screen shown in FIG. 11, the lines 61A to 63A indicating the positions of the tomographic images included in the scout image G0 are located near the chest in the schematic diagram 65 and are separated from the line 67 indicating the position of the landmark. Therefore, it can be known that the liver is not included in the imaging range of the scout image G0. However, in a case in which the operator looks at the display screen 60, it is possible to easily recognize which position on the scout image G0 should be set as the imaging range during the main imaging. Therefore, it is possible to easily specify the imaging range during the main imaging by using the scout image G0.

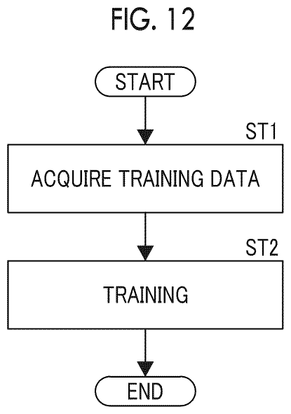

Hereinafter, the processing performed in the present embodiment will be described. FIG. 12 is a flowchart showing processing performed by the learning device according to the present embodiment. First, the information acquisition unit 21 acquires the training data from the image storage server 3 (step ST1). Next, the learning unit 23 constructs the derivation model 22A by training the CNN 35 using the training data (step ST2), and the processing ends.

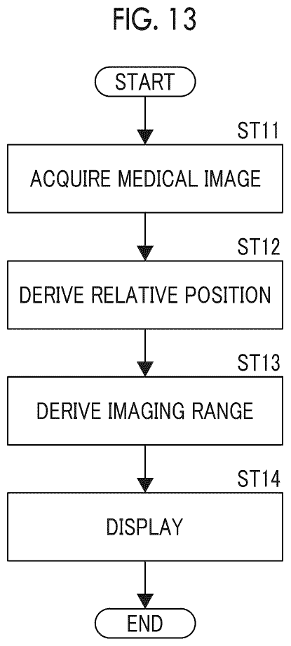

FIG. 13 is a flowchart showing processing performed by the image processing device according to the present embodiment. First, the information acquisition unit 21 acquires the scout image G0 as a processing target from the image storage server 3 (step ST11). Next, the position derivation unit 22 derives the normalized relative position of the processing target tomographic image in the interior of the body included in the scout image G0 (step ST12). Next, the range derivation unit 24 derives the imaging range of the scout image G0 (step ST13), the display controller 25 displays the display screen including the imaging range (step ST14), and the processing ends.

As described above, in the present embodiment, the position derivation unit 22 derives the normalized relative position, in the interior of the body, of at least one processing target tomographic image by using the derivation model 22A. Therefore, even in a case in which the processing target tomographic image does not include the target anatomical structure, the position of the target anatomical structure can be specified based on the relative position of the processing target tomographic image in the interior of the body. Therefore, it is possible to easily specify the imaging range during the main imaging using the processing target tomographic image such that the target anatomical structure is included.

In addition, by referring to the relative position, in the interior of the body, of the processing target tomographic image, it is possible to easily specify the processing target tomographic image including the desired anatomical structure based on the relative position of the desired anatomical structure in the interior of the body.

In addition, by deriving the normalized relative position in the interior the body for the tomographic images included in the three-dimensional images acquired by different imaging apparatuses, such as the CT image and the MRI image, it is possible to perform registration between the images acquired by different imaging apparatuses.

In this embodiment, each process is executed on an arbitrary computer. The arbitrary computer may execute these processes by means of a processor as hardware, a program as software, or a combination of the processor and the program. In such a case, the processor is configured to execute the various processes in this embodiment in cooperation with the program and may function as each unit or means in this embodiment. In addition, the order in which the processor executes these processes is not limited to the order described in this embodiment and may be changed as appropriate. The arbitrary computer may be a general-purpose computer, a computer for a specific purpose, a workstation, or any other system capable of executing each process.

The processor may be configured by one or more hardware, and the type of hardware is not limited. For example, the processor may comprise at least one of programmable logic devices such as CPUs (Central Processing Units), MPUs (Micro Processing Units), and FPGAs (Field Programmable Gate Arrays); dedicated circuits for performing specific processes such as ASICs (Application Specific Integrated Circuits); and other hardware such as a GPU (Graphics Processing Unit) and an NPU (Neural Processing Unit). The hardware may also be a combination of different types of hardware. When multiple hardware are configured to execute one or more processes of a processor, the said multiple hardware may exist in devices that are physically separate from each other, or in the same device. In any embodiment, the order of each process by the processor is not limited to the order described above and may be changed as appropriate. The hardware is configured by an electric circuit (circuitry) etc. that combines circuit elements such as semiconductor devices.

Furthermore, the program may be firmware or software such as microcode. The program may also be a group of program modules, each function of which may be performed by a processor configured to execute each of the program modules. The program may be program code or code segments stored on one or more non-transitory computer-readable media (e.g., storage media or other storage). The program may be stored in separate non-transitory computer-readable media located on devices that are physically separate from each other. The program code or code segments may represent any combination of procedures, functions, subprograms, routines, subroutines, modules, software packages, classes, instructions, data structures, or program statements. The program code or code segments may be connected to other code segments or hardware circuits by sending or receiving information, data, arguments, parameters, or memory contents.

In the above embodiment, it is explained that the image processing program 12A and a learning program 12B is stored (installed) in advance in the storage unit 13, but this is not limited to this. The image processing program 12A and the learning program 12B may be provided in a form recorded on a recording medium such as a CD-ROM (Compact Disc Read Only Memory), DVD-ROM (Digital Versatile Disc Read Only Memory), and USB (Universal Serial Bus) memory. In addition, the image processing program 12A and the learning program 12B may be provided in a form that the image processing program 12A and the learning program 12B are downloaded from an external device via a network.

The technology of this disclosure also extends to all types of program products. Program products include all types of products for providing programs. For example, program products include programs provided via networks such as the Internet, and non-temporary computer readable storage media such as CD-ROMs, DVDs, and USB memory devices that store programs.

Hereinafter, the supplementary notes of the present disclosure will be described.

Supplementary Note 1

An image processing device comprising: a processor, in which the processor is configured to: input at least one processing target tomographic image to a derivation model constructed by contrastive learning using a plurality of tomographic images acquired by imaging an interior of a body such that a specific anatomical structure is included, the derivation model being constructed by the contrastive learning so as to derive a normalized relative position in the interior of the body based on a relative reference position, which is determined in advance for the specific anatomical structure, in the interior of the body; and derive a normalized relative position of the at least one processing target tomographic image in the interior of the body via the derivation model.

Supplementary Note 2

The image processing device according to supplementary note 1, in which the reference position of the specific anatomical structure is a position of a landmark in the interior of the body.

Supplementary Note 3

The image processing device according to supplementary note 1 or 2, in which the derivation model is constructed by deriving a loss for matching a normalized relative position of the specific anatomical structure with the reference position, and training a learning target model through the contrastive learning so that the loss is decreased.

Supplementary Note 4

The image processing device according to any one of supplementary notes 1 to 3, in which the processor is configured to: convert the derived relative position into an absolute position.

Supplementary Note 5

The image processing device according to supplementary note 4, in which the processor is configured to: display a position of the specific anatomical structure based on the absolute position and a position of the processing target tomographic image.

Supplementary Note 6

A learning device comprising: a processor, in which the processor is configured to: train a learning target model through contrastive learning so as to derive, in a case in which a plurality of tomographic images including a specific anatomical structure is input, a normalized relative position in an interior of a body based on a relative reference position, which is determined in advance for the specific anatomical structure, in the interior of the body, to construct a derivation model that derives, in a case in which at least one processing target tomographic image is input, a normalized relative position of the at least one processing target tomographic image in the interior of the body.

Supplementary Note 7

The learning device according to supplementary note 6, in which the processor is configured to: input the tomographic images to the learning target model to derive at least one first relative position, which is normalized, in the interior of the body and further derive a second relative position, which is normalized, of the specific anatomical structure in a case in which the specific anatomical structure is included in the tomographic images; derive a first loss for matching the first relative position with the relative position in the interior of the body and a second loss for matching the second relative position with the reference position; and train the model so that the first loss and the second loss are decreased, to construct the derivation model.

Supplementary Note 8

An image processing method executed by a computer, the image processing method including: inputting at least one processing target tomographic image to a derivation model constructed by contrastive learning using a plurality of tomographic images acquired by imaging an interior of a body such that a specific anatomical structure is included, the derivation model being constructed by the contrastive learning so as to derive a normalized relative position in the interior of the body based on a relative reference position, which is determined in advance for the specific anatomical structure, in the interior of the body; and deriving a normalized relative position of the at least one processing target tomographic image in the interior of the body via the derivation model.

Supplementary Note 9

A learning method executed by a computer, the learning method including: training a learning target model through contrastive learning so as to derive, in a case in which a plurality of tomographic images including a specific anatomical structure is input, a normalized relative position in an interior of a body based on a relative reference position, which is determined in advance for the specific anatomical structure, in the interior of the body, to construct a derivation model that derives, in a case in which at least one processing target tomographic image is input, a normalized relative position of the at least one processing target tomographic image in the interior of the body.

Supplementary Note 10

An image processing program causing a computer to execute a procedure including: inputting at least one processing target tomographic image to a derivation model constructed by contrastive learning using a plurality of tomographic images acquired by imaging an interior of a body such that a specific anatomical structure is included, the derivation model being constructed by the contrastive learning so as to derive a normalized relative position in the interior of the body based on a relative reference position, which is determined in advance for the specific anatomical structure, in the interior of the body; and deriving a normalized relative position of the at least one processing target tomographic image in the interior of the body via the derivation model.

Supplementary Note 11

A learning program causing a computer to execute a procedure including: training a learning target model through contrastive learning so as to derive, in a case in which a plurality of tomographic images including a specific anatomical structure is input, a normalized relative position in an interior of a body based on a relative reference position, which is determined in advance for the specific anatomical structure, in the interior of the body, to construct a derivation model that derives, in a case in which at least one processing target tomographic image is input, a normalized relative position of the at least one processing target tomographic image in the interior of the body.

What is claimed is:

1. An image processing device comprising:
a processor,
wherein the processor is configured to:
input at least one processing target tomographic image to a derivation model constructed by contrastive learning using a plurality of tomographic images acquired by imaging an interior of a body such that a specific anatomical structure is included, the derivation model being constructed by the contrastive learning so as to derive a normalized relative position in the interior of the body based on a relative reference position, which is determined in advance for the specific anatomical structure, in the interior of the body; and
derive a normalized relative position of the at least one processing target tomographic image in the interior of the body via the derivation model.

2. The image processing device according to claim 1,
wherein the reference position of the specific anatomical structure is a position of a landmark in the interior of the body.

3. The image processing device according to claim 1,
wherein the derivation model is constructed by deriving a loss for matching a normalized relative position of the specific anatomical structure with the reference position, and training a learning target model through the contrastive learning so that the loss is decreased.

4. The image processing device according to claim 1,
wherein the processor is configured to:
convert the derived relative position into an absolute position.

5. The image processing device according to claim 4,
wherein the processor is configured to:
display a position of the specific anatomical structure based on the absolute position and a position of the processing target tomographic image.

6. A learning device comprising:
a processor,
wherein the processor is configured to:
train a learning target model through contrastive learning so as to derive, in a case in which a plurality of tomographic images including a specific anatomical structure is input, a normalized relative position in an interior of a body based on a relative reference position, which is determined in advance for the specific anatomical structure, in the interior of the body, to construct a derivation model that derives, in a case in which at least one processing target tomographic image is input, a normalized relative position of the at least one processing target tomographic image in the interior of the body.

7. The learning device according to claim 6,
wherein the processor is configured to:
input the tomographic images to the learning target model to derive at least one first relative position, which is normalized, in the interior of the body and further derive a second relative position, which is normalized, of the specific anatomical structure in a case in which the specific anatomical structure is included in the tomographic images;
derive a first loss for matching the first relative position with the relative position in the interior of the body and a second loss for matching the second relative position with the reference position; and
train the model so that the first loss and the second loss are decreased, to construct the derivation model.

8. An image processing method executed by a computer, the image processing method comprising:
inputting at least one processing target tomographic image to a derivation model constructed by contrastive learning using a plurality of tomographic images acquired by imaging an interior of a body such that a specific anatomical structure is included, the derivation model being constructed by the contrastive learning so as to derive a normalized relative position in the interior of the body based on a relative reference position, which is determined in advance for the specific anatomical structure, in the interior of the body; and
deriving a normalized relative position of the at least one processing target tomographic image in the interior of the body via the derivation model.

9. A learning method executed by a computer, the learning method comprising:
training a learning target model through contrastive learning so as to derive, in a case in which a plurality of tomographic images including a specific anatomical structure is input, a normalized relative position in an interior of a body based on a relative reference position, which is determined in advance for the specific anatomical structure, in the interior of the body, to construct a derivation model that derives, in a case in which at least one processing target tomographic image is input, a normalized relative position of the at least one processing target tomographic image in the interior of the body.

10. A non-transitory computer-readable storage medium that stores an image processing program causing a computer to execute a procedure comprising:
inputting at least one processing target tomographic image to a derivation model constructed by contrastive learning using a plurality of tomographic images acquired by imaging an interior of a body such that a specific anatomical structure is included, the derivation model being constructed by the contrastive learning so as to derive a normalized relative position in the interior of the body based on a relative reference position, which is determined in advance for the specific anatomical structure, in the interior of the body; and
deriving a normalized relative position of the at least one processing target tomographic image in the interior of the body via the derivation model.

11. A non-transitory computer-readable storage medium that stores a learning program causing a computer to execute a procedure comprising:
training a learning target model through contrastive learning so as to derive, in a case in which a plurality of tomographic images including a specific anatomical structure is input, a normalized relative position in an interior of a body based on a relative reference position, which is determined in advance for the specific anatomical structure, in the interior of the body, to construct a derivation model that derives, in a case in which at least one processing target tomographic image is input, a normalized relative position of the at least one processing target tomographic image in the interior of the body.
